(19) United States
(12) Patent Application Publication
(10) Pub. No.: US 2026/0279556 A1
(43) Pub. Date: Sep. 17, 2026

(54) CLOUD-BASED CONVERSATIONAL ARTIFICIAL INTELLIGENCE PLATFORM AND METHOD FOR AI-DRIVEN MULTI-SYSTEM COMMUNICATIONS BETWEEN HEALTHCARE PRACTICES

(71) Applicant: Reena Gajjar, Scottsdale, AZ (US)

(72) Inventor: Reena Gajjar, Scottsdale, AZ (US)

(21) Appl. No.: 19/564,039

(22) Filed: Mar. 11, 2026

Related U.S. Application Data
(60) Provisional application No. 63/771,055, filed on Mar. 13, 2025.

(51) Int. Cl. G16H 40/20; G06N 3/045; G10L 15/18; G16H 10/60

(52) U.S. Cl. CPC G16H 40/20; G06N 3/045; G10L 15/1815; G16H 10/60

(57) ABSTRACT

A cloud-based, dental-first conversational AI platform and computer-implemented method autonomously orchestrates communications among multiple cross-platform software systems within a dental healthcare ecosystem. The platform includes a conversational AI agent utilizing voice recognition and natural language processing to interact with patients in real time while concurrently coordinating scheduling, insurance verification, referral management, and inventory control. The AI agent maintains conversational context, communicates with external systems via secure APIs, and exchanges structured task data with other AI agents to optimize workflow efficiency, reduce administrative burden, and improve patient care. The system is extensible to broader medical healthcare environments.

### REFERENCE TO RELATED APPLICATIONS

This application is a Non-provisional Patent Application claiming priority to Provisional Patent Application Ser. No. 63/771,055 filed Mar. 13, 2025 and titled PLATFORM AS A SERVICE (PAAS) AND METHOD FOR AI-DRIVEN MULTI-SYSTEM COMMUNICATIONS BETWEEN HEALTHCARE PRACTICES, the disclosure of which is incorporated herein in its entirety by reference.

### FIELD OF THE INVENTION

The present invention relates generally to cloud-based artificial intelligence systems and, more particularly, to a dental-first conversational artificial intelligence (AI) platform that autonomously orchestrates interactions among multiple cross-platform healthcare systems to optimize administrative workflows, enhance patient care, and improve operational efficiency, consistent with USPTO Patent Eligibility Guidance (PEG) principles for computer-implemented inventions. The systems described herein may include but not limited to:
practice management systems (PMS)
third-party healthcare Saas (Software as a Service) tools inventory control systems
insurance verification interfaces
cross-practice referral networks

### BACKGROUND OF THE INVENTION

Dental practices operate within highly fragmented software environments, typically relying on multiple independent systems for patient management, scheduling, radiographic imaging, insurance verification, inventory control, laboratory coordination, and referral management. These systems rarely interoperate seamlessly, requiring extensive manual data entry, redundant workflows, and staff-mediated coordination.

Administrative inefficiencies consume significant clinical and non-clinical resources. Insurance verification often requires prolonged telephone interactions with insurance carriers. Referral coordination requires repeated back-and-forth communications between practices. Scheduling changes necessitate manual updates across disparate platforms. These inefficiencies delay care, increase operating costs, and degrade the patient experience.

One of the major inefficiencies in healthcare practices is the fragmentation of workflows. Dental offices typically use multiple software systems to manage different aspects of patient care, including patient management systems (PMS), radiograph analysis, billing, and insurance verification. Each time a change is made to a patient's file, staff members must manually enter data across multiple systems, leading to redundant work and an increased likelihood of errors. This manual process not only consumes valuable time but also reduces overall efficiency and productivity within the practice.

Another critical issue faced by healthcare providers is the significant delay in insurance verification. Insurance-related tasks require doctors and administrative staff to spend an excessive amount of time on the phone with insurance carriers to verify patient coverage. Studies indicate that 82% of dental offices experience delays in obtaining insurance verifications over the phone, creating bottlenecks that hinder patient scheduling and treatment planning. These delays can result in postponed procedures, increased administrative costs, and frustration for both patients and providers.

Additionally, referral inefficiencies pose challenges in coordinating patient care between providers. When a patient requires a referral to a specialist, setting up an appointment with the referred doctor can be a complex and time-consuming process. The referring and referred doctors must communicate to ensure proper scheduling while also accommodating the patient's availability. Further, follow-ups between referring and referred doctors are often not streamlined, leading to gaps in care coordination and potential miscommunication.

Given these challenges, there is a growing need for an AI-powered platform to automate and streamline key administrative tasks in healthcare practices. Such a platform could facilitate automated scheduling of patient appointments, referral coordination, insurance verification, and medical record management. By integrating artificial intelligence, healthcare providers could significantly reduce manual data entry, minimize administrative burdens, and improve overall workflow efficiency. The implementation of an AI-driven solution would enhance patient care by allowing healthcare professionals to focus more on clinical tasks rather than administrative duties, ultimately leading to better patient outcomes and a more efficient healthcare system. A platform as described is also desirable in that communications with an insurance company, a referred medical practice, and a radiographic vendor, may occur in real time and simultaneously/autonomously-even while intelligently conversing with a respective patient or even with multiple patients.

### SUMMARY OF THE INVENTION

The present invention provides a cloud-based conversational AI platform and computer-implemented method that autonomously orchestrates communications among a plurality of cross-platform systems within a dental healthcare ecosystem for treating a patient in need of dental care.

The platform (which may also be referred to as a Platform as a Service (or PAAS) as Software as a Service (Saas)) may include a computer system that may include a conversant AI agent which may be referred to in use as “Reena” with the personalized familiarity that users have with AI devices such as Alexa, Siri, Google Assistant, and the like.

Implementation of the invention described in this application, e.g., the application of “Reena” across the many aspects of a healthcare industry ecosystem will have industrywide benefits that are completely disruptive and non-obvious. In other words, the invention recited and disclosed below is an absolute disruption to an industry that has not seen such efficiency literally for decades. Such a disruption is unexpected to say the least.

In a preferred embodiment, the platform includes a conversant AI agent—referred to herein as “Reena”—that utilizes voice recognition and natural language processing to converse with patients, dental providers, and staff in real time, while simultaneously communicating with external systems including practice management systems (PMS), insurance carriers, dental laboratories, radiographic analysis platforms, inventory control systems, and referral dental or medical practices.

The AI agent autonomously transforms unstructured natural language input into structured, machine-executable task data, enabling automated scheduling, insurance verification, referral coordination, record transfer, and inventory management without manual intervention.

While optimized for dental practices, the platform is extensible to medical healthcare workflows using the same architectural principles.

Therefore, a general object of this invention is to provide a cloud-based AI platform and computer-implemented method that autonomously orchestrates interactions between a plurality of cross-platform systems within a predetermined healthcare ecosystem seeking to treat a patient.

Another object of this invention is to provide an AI platform, as aforesaid, that facilitates automated scheduling of patient appointments, referral coordination, insurance verification, communication with other practice providers within a healthcare ecosystem system, and medical record management.

Still another object of this invention is to provide an AI platform, as aforesaid, that includes an AI agent capable of communicating simultaneously with a live patient while in real time data and oral communication with AI agents of other healthcare providers associated with the same patient.

A further object of this invention is to provide an AI platform, as aforesaid, that improves the efficiency of a healthcare practice by an exponential factor.

Still another object of this invention is to provide an AI platform, as aforesaid, that reduces the number of staff required to operate a healthcare practice.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a diagram illustrating a Dental healthcare ecosystem integrated by the conversational AI platform according to the present invention;

FIG. 2 is a block diagram of a system architecture for implementing the platform of FIG. 1;

FIG. 3 is a flowchart illustrating real-time AI agent communication workflows according to the present invention;

FIG. 4 is a logic diagram illustrating an Interactive Voice Recognition (IVR) communication with a Practice Management System (PMS) according to the present invention;

FIG. 4a is another logic diagram illustrating the natural language processing module of FIG. 4 in greater detail according to the present invention;

FIG. 5 is a flowchart illustrating a dental referral workflow using AI-to-AI communication;

FIG. 6 is a block diagram illustrating how an entire industry ecosystem is improved according to the present invention; and

FIG. 7 is a block diagram illustrating the use of Interactive Voice recognition (IVR) is utilized across the entire industry ecosystem according to the present invention.

### DETAILED DESCRIPTION OF THE PREFERRED EMBODIMENTS

A cloud-based AI system 10 and computer-implemented method according to the present invention will now be described with reference to the accompanying drawings. The system autonomously orchestrates interactions between a plurality of cross-platform systems within a predetermined healthcare ecosystem. The cloud-based and Internet accessible system 10 essentially sets up a computer-accessible “platform” 10a. It will be understood that a person of ordinary skill in the art may find the terms system 10 and platform 10a to be essentially synonymous and so either term may be accurately used in the detailed description below and in the claims reciting the present invention.

### System Architecture (Dental-First)

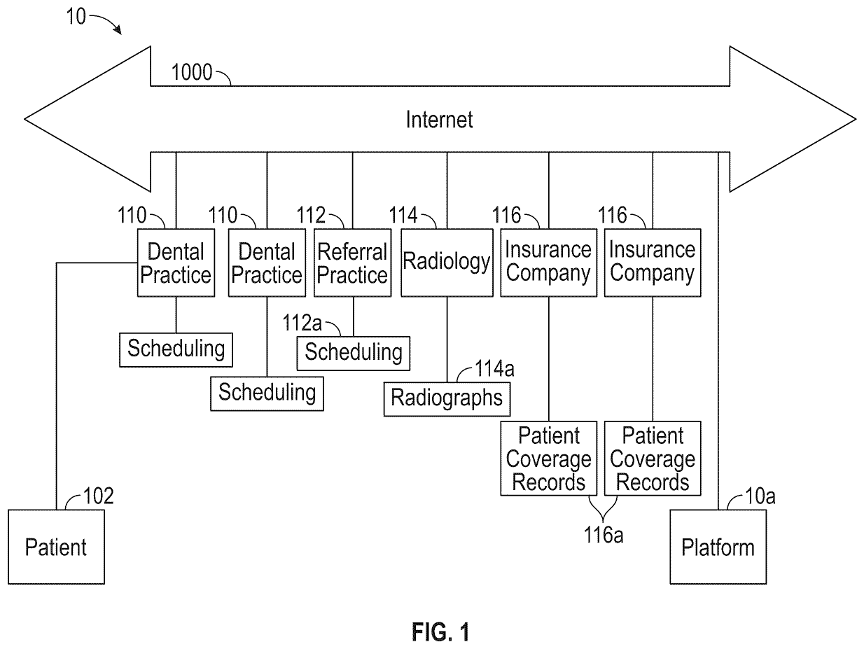

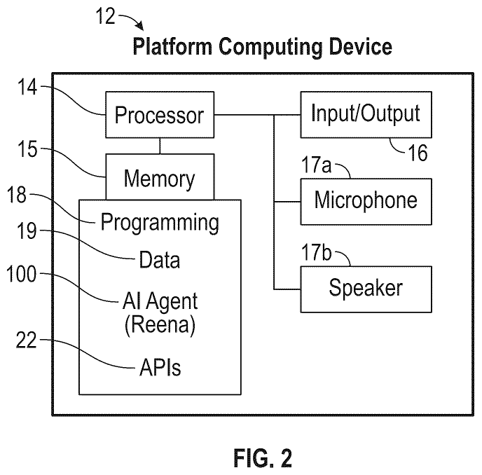

The platform 10a comprises a computing device 12 in data communication with the Internet 1000 and a cloud-based artificial intelligence environment (FIG. 1). Preferably, the computing device 12 includes one or more processors 14, non-transitory memory 15, and input/output components 16 which may include a microphone 17a, a speaker 17b, a display (with or without touchscreen technology), a mouse, and network interfaces (FIG. 2). In an embodiment, the computing device 12 may be a server, workstation, or other device capable of storing programming instructions 18 and data 19 and having access to the Internet 1000. In an embodiment, there may be multiple computing devices 12, each having one or more processors 14 and configured as described above. This embodiment would be illustrative of a plurality of practice management systems across one or more healthcare industries implementing the invention as will be described below.

In one embodiment, the platform 10a may be characterized as an orchestration engine executed by the one or more processors 14 and stored at least in part within the non-transitory memory 15 of the computing device 12. The orchestration engine is a dedicated control and coordination module operatively coupled to one or more conversational artificial intelligence agents 100 and and configured to manage execution of multi-step healthcare administrative workflows across a plurality of heterogeneous software platforms. The orchestration engine receives structured task data generated by the natural language processing subsystem of a conversational AI agent and decomposes the structured task data into one or more executable workflow actions (each of which will be discussed in greater detail later). Each workflow action corresponds to a specific operation to be performed by an external system, including practice management systems, scheduling systems, inventory systems, insurance verification interfaces, or referral practice systems.

In In a further aspect, the orchestration engine is configured to sequence, parallelize, and monitor execution of the workflow actions using asynchronous task execution logic, state management data, and error-handling routines. The orchestration engine maintains workflow state information that tracks execution progress, system responses, and dependency conditions between workflow actions, thereby enabling concurrent backend processing while a conversational AI agent continues to interact with a patient or practice user in real time. The orchestration engine further includes interface logic for invoking application programming interfaces (APIs), generating machine-readable messages, and routing responses back to the conversational AI agent for natural language response generation. Accordingly, the orchestration engine provides a concrete technical mechanism for coordinating distributed software operations and improving overall system efficiency within the dental healthcare ecosystem.

### Software as a Service (SaaS)

In a critical aspect, a substantial portion of the AI logic, including natural language processing models and dialogue management engines, executes remotely in the cloud, enabling continuous updates and scalability across multiple dental practices. When software or data is “in the cloud,” it means it runs on powerful, remote servers in massive data centers, managed by a provider, and accessed over the internet, not directly on a user's personal computer or local server. More particularly, the data and software described herein may be physically stored (i.e., may “live”) on servers in large data centers owned by companies like Amazon (AWS), Microsoft (Azure), or Google (GCP). Connection to these resources is made via the Internet 1000 (which refers to a wide area network) and not just via a local network (which may be hardwired within an office building). Resources accessed and used in the cloud actively managed by large commercial entities and experts, making them more efficient and reliable to hundreds or even thousands of users simultaneously. Still further, cloud applications usually involve automatic and regular updates and data protection that occur seamlessly and autonomously. Accordingly, the present invention may be characterized, accessed, and referred to as a Software as a Service (Saas).

Much of this universal access is made possible through the use of APIs 20. An API (Application Programming Interface) is a set of rules and definitions (typically stored in the cloud) that enables different software programs to communicate and exchange data with each other. It acts as a messenger or intermediary, allowing applications to request data or functionality from other systems without needing to know the internal workings of those systems.

As will be discussed later in more detail, the present invention will utilize various subsets of artificial intelligence (referred to as “AI Subsets” 20) such as machine learning 30, natural language processing 40, and large language models 50 ((FIG. 4). More particularly, machine learning (ML) 30 is a subset of AI that enables systems to learn from data and improve performance without explicit programming. Machine Learning (ML)) 30 is a subset of AI that enables systems to learn from data and improve performance without explicit programming instructions. Further, another subset of AI that may be utilized prominently in the present invention is Natural Language Processing (NLP) 40. NLP refers to the ability of an AI system to understand, interpret, and generate human language. Both of these subsets may be utilized in making the AI agent conversant with humans as well as other AI agents, as will be described in more detail later. In the present invention, the NLP 40 may be further described in greater detail as including a natural language processing subsystem 42 and a speech processing subsystem 44.

The concepts of machine learning and natural language processing are closely related and include considerations of another aspect of artificial intelligence referred to as large language models (LLM) 50. More particularly, a Large Language Model (LLM) 50 is a specialized subset of Machine Learning (ML) 30 that falls under Natural Language Processing (NLP) 40. These subsets of artificial intelligence that may be utilized in the present invention and which may be called using respective APIs 22 may be summarized as follows:
1. Machine Learning (ML) 30: This is a branch of AI that enables systems to learn patterns from data and make predictions without being explicitly programmed.
2. Deep Learning (DL): A subset of ML that uses artificial neural networks, particularly deep neural networks, to model complex patterns and relationships in data.
3. Natural Language Processing (NLP) 40: A specialized field within ML that focuses on enabling computers to understand, interpret, and generate human language. In an embodiment, the NLP 40 may include a natural language processing subsystem 42 and a speech subsystem 44, both of which will be described in greater detail later.
4. Large Language Model (LLM) 50: A subcategory of NLP that consists of deep learning models trained on massive datasets to generate human-like text. Examples include GPT (like ChatGPT), BERT, and T5.

In the present invention, the AI concepts introduced above and the AI modules accessed by particular structures described below are leveraged to make the AI agent 100 more conversant, more accurate, more efficient, and faster. The details regarding how artificial intelligence modules can make the AI agent conversant with a human person as well as with another AI agent 100 are more understood with reference to large language models as outlined below:
Use of Deep Learning: LLMs employ transformer architectures, such as the Transformer neural network, to process and generate language.
Pretrained on Massive Datasets: These models learn language patterns by training on large corpora of text data.
Fine-Tuning for Specific Tasks: LLMs can be adapted for tasks like translation, summarization, and conversational AI.
Generative Capabilities: They can produce coherent, contextually relevant text responses.

The AI platform 10a according to the present invention serves as a communication and connection platform to other “Reena's” or systems within the ecosystem that the practice selects. Communication is transferred by human voice or to conversant AI and back or system-to-system via API as needed. The computing device itself need not be intelligent on its own. The artificial intelligence agent 100 known as “Reena” controls all actions initiated by a user, preferably through a back-and-forth exchange of voice recognition and natural language processing paradigms. The vast majority of “Reena” lives in the “cloud.” As first introduced above, the computing device 12 serves as the platform for input and output. The computing device 12 on which the platform 10a is created may include a controller or processor 14, a non-volatile memory 15, and input and output devices such as a mouse. The computing device, of course, is in data communication with the Internet 1000 to facilitate communications across associated platforms also connected to the Internet 1000.

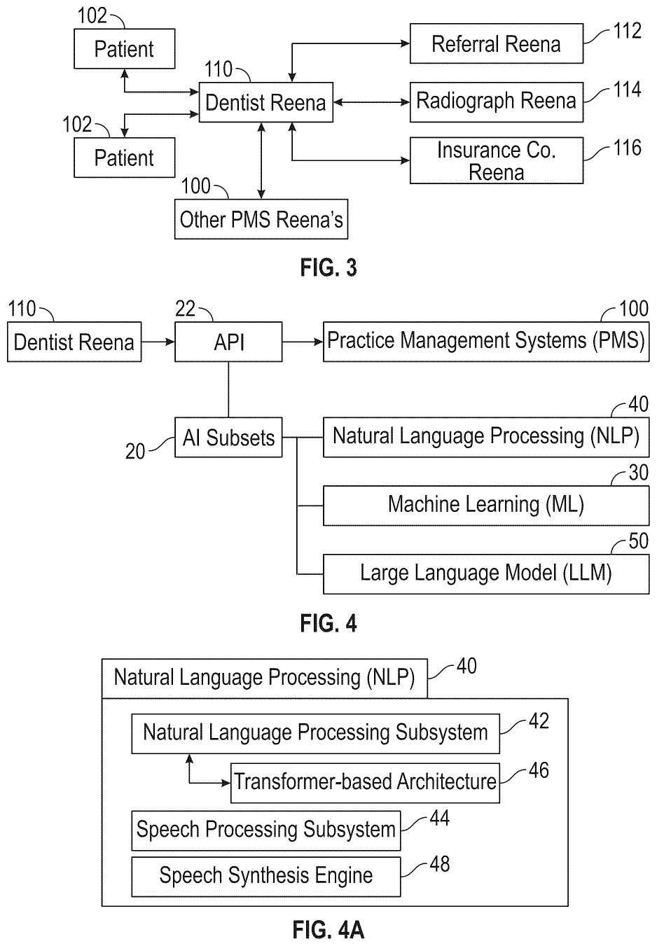

Preferably, the system 10 is generated using open-source API technology from vendors which the office chooses to use, (for instance, radiograph analysis, payment systems, Practice Management Systems (PMS), inventory control, pharmacies, vendors, specialists and others) (FIGS. 3, 5, and 6). The platform 10a according to the present invention will be referred to herein as “Reena”. Reena connects to the respective vendor and issues the commands needed for the action to be executed in a secure manner. Reena can be configured for any environment that is specialized, recognizing scientific or procedural jargon specific to the functions it needs to commit actions in a specific environment. It is important to note that Reena is technology neutral and a neutral instance of Reena may be referred to as communicating with another Practice Management System 100 that is also operating an instance of Reena. More particularly, it will be understood that the phrase “Practice Management System 100 operating an instance of Reena” intentionally utilizes the reference character 100 along with generic references to a conversant AI agent 100. That means that it can connect to, for instance, any number of Practice Management Systems 100 in the market as long as they have an associated API interface engine 22. An API, which stands for “Application Programming Interface,” is a set of rules or protocols that allows different software applications to communicate with each other, exchanging data and functionality by acting as a middle ground between them; essentially, it's a way for one program to request and receive information from another program.

Since Reena is located (at least partially) in the “cloud” the latest version is always running. In the event that the device needs to be updated, Reena can initiate an update routine located on the device. The agent also continuously checks for malfunctions with every point of contact in its universe. This may include the device itself or the connections between third parties. Broken connections may be repaired via updates to the cloud-based system 10.

### Conversational AI Agent

The AI agent “Reena” 100 serves as the primary interface between human users and backend systems. Reena is configured to converse naturally with dental patients 102 and staff, interpret spoken and textual language, maintain conversational context, and autonomously execute dental administrative workflows. It will be understood that the AI Agent may be referred to herein as the conversational AI agent, the AI agent, or Reena, each phrase being represented by the reference 100. Therefore, the context from which the AI agent “Reena” is used or accessed can be illustrated to communicate said context. For instance, the “Reena” platform customized to the dentistry-first contact may be referred to in the illustrations as “Dentist Reena” 110. By contrast and as will be described in greater detail later, the platform may be customized and referred to as “Referral Reena 112,” “Radiograph Reena 114,” “Insurance Company Reena 116,” or with other Practice Management Systems 100 in general (i.e., other instances of Reena) (FIG. 3). It will be understood that similar terminology will utilize the same reference characters. For instance, “Dentist Reena” and a “Dental Practice” will both utilize reference character 110; “Referral Reena” and “Referral Practice” will utilize reference character 112; “Radiograph Reena” and “Radiology” will both utilize reference character 114; “Insurance Company Reena” and “Insurance Company” will both utilize reference character 116.

### Natural Language Processing Subsystem

The platform 10a includes an NLP subsystem 42 executed by one or more processors, comprising: 1. Automatic Speech Recognition (ASR) converting audio input into text using dental-specific vocabularies. 2. Linguistic preprocessing: tokenization, normalization, lemmatization, and abbreviation expansion. 3. Intent classification via transformer-based deep learning models trained on dental workflow data. 4. Named Entity Recognition (NER) extracting dental entities such as procedure types, tooth identifiers, radiograph references, insurance carriers, and provider identifiers. 5. Conversational state management storing task progress and unresolved parameters. 6. Natural Language Generation (NLG) producing context-appropriate spoken or textual responses. Each of these categories will be described in greater detail below.

In one embodiment, the natural language processing (NLP) subsystem 42 of the conversational AI agent operates as a modular, domain-adapted language understanding and generation pipeline executed across distributed computing resources. Incoming human speech signals are first processed by an automatic speech recognition (ASR) engine configured to produce time-aligned phoneme and token streams. These token streams are ingested by a domain-specialized language understanding module that applies a transformer-based neural network architecture comprising multiple self-attention layers trained to identify semantic intent, domain-specific entities, contextual dependencies, and task constraints. The NLP subsystem performs joint intent classification and entity extraction using a multi-head attention mechanism, enabling the system to simultaneously determine user objectives (e.g., scheduling, referral initiation, insurance inquiry) while extracting structured data elements such as procedure codes, provider identifiers, appointment durations, anatomical references, and insurance plan descriptors. Extracted entities are normalized against ontology tables maintained within the healthcare ecosystem, including standardized dental procedure vocabularies, insurance nomenclature, and practice-specific scheduling rules, thereby converting unstructured natural language into machine-actionable command structures.

In one embodiment, the natural language processing (NLP) subsystem 42 implemented by the conversational artificial intelligence agent 100 employs a transformer-based neural network architecture 46 configured to perform joint intent classification and named-entity recognition within a single inference pass. The transformer architecture 46 utilizes multiple stacked self-attention layers, each comprising multi-head self-attention mechanisms that enable the model to evaluate contextual relationships between words, phrases, and domain-specific terminology appearing in a user utterance. By processing multiple attention heads in parallel, the NLP subsystem 42 can simultaneously capture syntactic structure, semantic meaning, and task-relevant cues, allowing the system 10 to accurately determine a user's intended action while extracting relevant dental-specific entities such as procedure types, provider identifiers, scheduling constraints, and insurance descriptors. This joint modeling approach reduces computational latency, improves inference accuracy, and enables real-time conversational interaction while generating structured task data suitable for downstream orchestration across healthcare software platforms.

In a critical aspect, the NLP subsystem 42 is trained and continuously refined using a domain-adaptive learning pipeline specific to a given medical ecosystem, such as dentistry. During an initial training phase, a large language model is pretrained on general-purpose corpora and subsequently fine-tuned using curated datasets derived from dental practice management systems, anonymized clinical notes, appointment transcripts, insurance communications, radiology reports, and referral correspondence. This fine-tuning process incorporates supervised learning with labeled intents and entities unique to dental workflows (e.g., crown placement, implant staging, periodontal charting, radiograph types, CDT codes), as well as reinforcement learning from task completion outcomes within live practice environments. Vocabulary embeddings are dynamically updated to reflect evolving terminology, vendor-specific procedure descriptors, and practice-defined shorthand, enabling the NLP subsystem to disambiguate clinically similar phrases and adapt to localized linguistic patterns. As a result, the conversational AI agent 100 is capable of maintaining contextual continuity across multi-turn dialogues, concurrently engaging with human users and external systems, and generating context-aware natural language responses or structured API calls that are semantically consistent with the operational constraints and regulatory requirements of the dental healthcare ecosystem.

The NLP subsystem 42 transforms unstructured speech into structured task data used to invoke API-based system actions.

### Speech Processing Subsystem

In an embodiment, the platform 10a includes a speech processing subsystem 44 operatively connected to the conversational artificial intelligence agent and executed by one or more processors of the computing system. The speech processing subsystem 44 is configured to manage bidirectional audio communication between human users and the conversational artificial intelligence agent 100 and comprises one or more of: an automatic speech recognition (ASR) engine 48, a speech signal pre-processing module, and a speech synthesis engine (all of which are referred to with reference character 48). The ASR engine 48 receives analog or digital audio signals captured from a microphone, telephone interface, or network audio stream and converts the received speech into time-aligned textual token data suitable for downstream processing by the natural language processing (NLP) subsystem 42. The speech signal pre-processing module performs noise reduction, speaker segmentation, and acoustic feature extraction to improve recognition accuracy across varying acoustic environments, including operatory rooms and telephone networks.

In a further aspect, the speech processing subsystem 44 is configured to generate synthesized speech output using the speech synthesis engine 48, which converts machine-generated textual responses into audible speech delivered via a speaker, telephone line, or network audio interface. The speech processing subsystem 44 operates in coordination with the NLP subsystem 42 to support real-time, multi-turn conversational interactions, including simultaneous speech input from a patient 102 and speech output generated by the conversational artificial intelligence agent 100. The speech processing subsystem 44 further enables autonomous voice-based interactions with external systems, including interactive voice response (IVR) systems associated with insurance carriers or healthcare vendors, by programmatically generating spoken prompts and interpreting spoken responses without human intervention. Accordingly, the speech processing subsystem 44 provides a foundational technical component that enables natural language communication, real-time responsiveness, and concurrent human-to-machine and machine-to-machine voice interactions within the healthcare ecosystem.

### Conversational State and Task Orchestration

A dialogue management engine evaluates conversational state to determine whether sufficient parameters exist to execute a task. Reena is configured to generate follow-up prompts as needed. When sufficient data is present, Reena initiates concurrent backend operations while continuing real-time patient conversation.

### Cross-Platform and AI-to-AI Communication

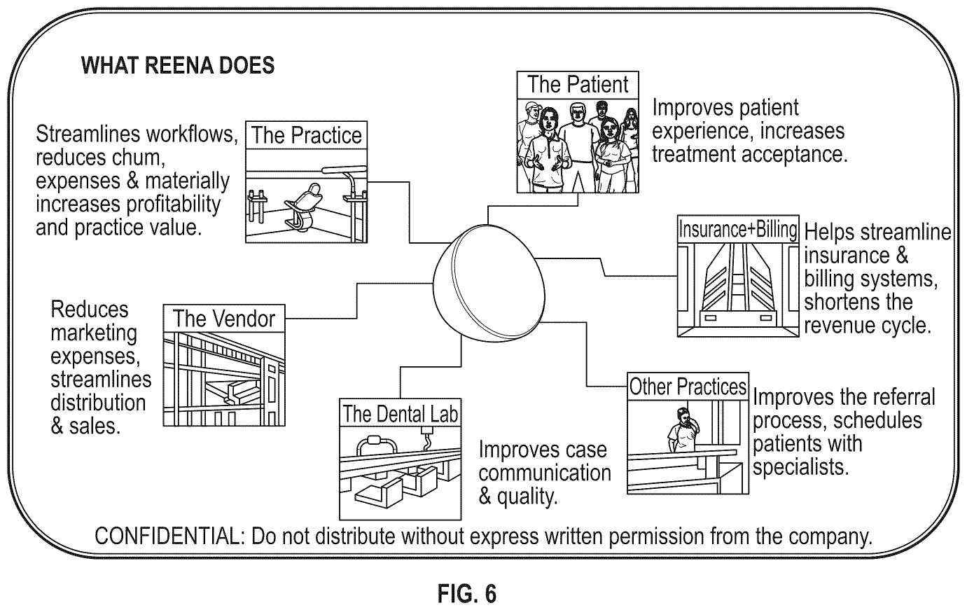

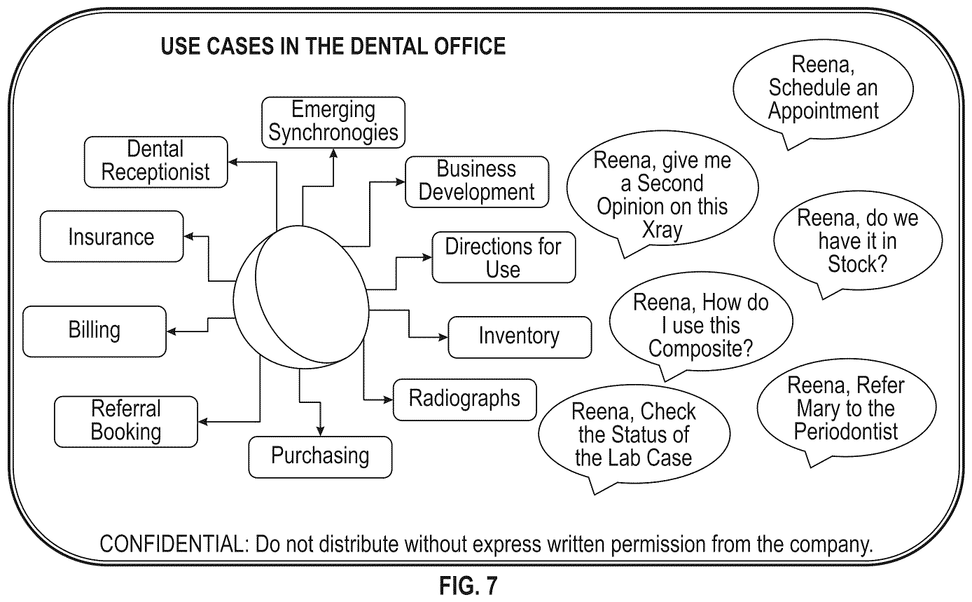

Reena communicates with external dental systems using secure APIs 22. For instance, in referral scenarios, Reena exchanges machine-readable semantic task messages with another instance of Reena operating at a referral practice 112, enabling autonomous negotiation of appointment availability 112a, insurance coverage 116a, and radiograph transfer 114a. With multiple instances of Reena operating at various autonomous dental practices, measures or surveys patient satisfaction or experience will increase favorably based on consistency, efficiency, and multi-tasking advantages while simultaneously decreasing labor/employee costs. Further, each individual dental practice may operate multiple instances of Reena “in the background”, overnight, or in real-time to evaluate and even make automatic purchasing decisions regarding inventory, negotiating billing inquiries or claims management, and even spending time engaging in business development and customer acquisition communications. The many practical “use cases” are illustrated in FIGS. 6 and 7. Use cases specific to the dental industry will be described below.

### Dental Use Cases

Automated dental scheduling, radiograph retrieval and analysis.
Insurance verification via IVR and NLP.
Inventory control for dental procedures.
Autonomous referral coordination between dental specialists.
   All workflows occur with minimal staff involvement and in real time.

There are many practical use cases that illustrate the practical uses of the present invention and that prove that the present invention includes “something more” than a mere abstract idea or abstract usage of artificial intelligence models or paradigms. In a critical distinction and in many of the following use cases, it is multiple iterations of AI agents (i.e., multiple Reena's being operational across multiple practice management systems or industries) that are interacting automatically, in real-time, and silently amongst one another as opposed to an AI agent merely responding to a human asking a question. In other words, it may be one instance of Reena asking a question to another instance of Reena. Multiple such use cases are profiled below:

### Use Case 1: Setup

The system, i.e., the platform 10a, is placed in a dental office 110 in sufficient numbers to interact with the humans in the office. It has been configured via human or AI Agent, to integrate securely with the practice's technology vendors, inventory control systems, other professional office practice management systems in which the practice is in a referral or partnership agreement and the local PMS. This may include advanced set up of passwords, access codes, or the like.

A configuration file may then be generated to create the ecosystem to this instance of Reena. That file is then uploaded to the platform 10a. In use, the system autonomously locates the related systems and then automatically and securely integrates with all systems named in the configuration file, vastly simplifying the integration process. Practically speaking, this may include calls to respective APIs 22 which enable intelligent communications between respective systems within the respective ecosystem or practice area.

For instance and as illustrated in FIGS. 6 and 7, upon receiving the credentials for the local PMS, the conversant AI agent referred to as Reena connects to all schedules, billing information, patient records and practice financial systems, among others. More particularly, consider if the practice needs a specific piece of equipment for a planned procedure, for instance, an implant. Reena then automatically checks with the practice's inventory control system (which may or may not be a third-party technology) to ensure that the needed implant is in stock. If not, Reena connects to the implant vendor in real time and orders the necessary implant to be delivered prior to the scheduled procedure. In short, Reena integrates scheduling, billing, obtaining patient records, and ordering equipment specific to a patient procedure in real time, autonomously, and with an efficiency that saves both time and money, minimizes error, and improves patient and staff satisfaction.

### Use Case 2: Radiographs

In a dental office, the platform 10a is installed or accessed in the cloud where needed to interact with the humans in the office using secured Wi-Fi or Bluetooth networking technology and voice recognition technology native to Reena.

A patient is in the operatory. A provider asks Reena to pull up the radiographs for the patient and highlight any areas of concern on the local display, most likely a smart television.

As shown in FIG. 1, Reena automatically connects it real-time and without specific prompting or instruction (other than requesting Reena to pull up respective radiographs for the patient) via API to the third-party system the practice has selected to analyze radiographs, connects to the local PMS, finds the radiographs requested and displays them as dictated by the radiograph analysis engine where requested. More particularly, obtaining and displaying patient records may involve Reena communicating with multiple patient management systems before obtaining and then displaying the records that are responsive to the live dentists oral request—all while a patient is live in the dental chair

### Use Case 3: a Referral

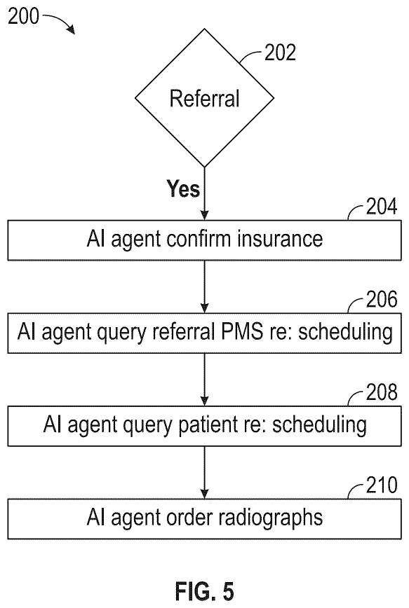

With reference to FIG. 5, the “referral” use case 200 is illustrated in more detail.

At step 202, if the dentist decides that a patient needs to be referred to an oral surgeon to whom he regularly refers patients, the conversant AI agent 100 will connect to a corresponding AI agent running at the office of the oral surgeon. As shown at step 204, the AI agent 100 may also autonomously and simultaneously connect with the patient's insurance company to confirm insurance coverage for the referral. The dentist describes the nature of the issue he is referring. At step 206, the dentist's Reena then automatically and in real-time reaches out via internet to the oral surgeon's instance of Reena and queries it for a few available times. More particularly, the surgeon's Reena analyzes the surgeon's schedule, looking for possible appointment times that are long enough for the procedure, when any needed staff is required, and returns the information to the dentist's instance of Reena. The “scheduling options” may be exchanged by the two instances of Reena and presented to the patient—who may still be in the referring dentist chair in real-time. The dentist Reena may conversationally present the options to the patient as indicated at step 208 and a firm date may be negotiated.

Then, as shown in step 210, once the appointment is booked, the dentist's Reena automatically sends the patient record, imaging and the dentist's notes to the surgeon's Reena, who then connects the information to the new patient record that it has created for the patient. The dentist's records where, appropriate, indicate the facts of the case as they stand. This may also automatically prompt obtaining respective radiographs and verification of insurance coverage and the procedures described above. In addition, Reena can document the multiplicity of actions to the patient's records autonomously and simultaneously.

Both instances of Reena then communicate according to the requirements dictated by the practice, for instance, a text message confirming the time and location of the surgeon's office to the patient.

Further, if the patient wants to know any information, for instance, the cost, then Reena may query the appropriate systems and deliver the appropriate response. The surgeon's Reena already knows if the patient has valid insurance because that is transmitted with the new patient file. If the patient agrees, the appointment is made at the oral surgeon.

Once the patient receives the treatment required by the oral surgeon, the surgeon's Reena autonomously communicates the necessary information to its counterpart in the dentist's office and schedules a follow up in the same manner that it made the appointment at the oral surgeon's office and sends a message via email or text to all concerned parties with the information they need to know.

One result is a more satisfied patient who doesn't have to spend time going back and forth trying to get appointments, provide information or other clerical issues. The patient shows up at the oral surgeon as scheduled, receives the necessary treatment and receives the date and time for the follow ups as required. All of this occurs seamlessly (including all documentation, billing, and insurance requirements) and with minimal doctor or staff involvement.

Accordingly, staff time is dramatically reduced as Reena has taken care of appointments, referral communication, payment information, and inventory control. This makes the practice more profitable and may result in lower cost care, and more time for the dentist, which may result in better patient outcomes.

### Use Case 4: Autonomous Cross-Selling Opportunities

With continued reference to the dental ecosystem, a patient calls the dentist's office. The phone is answered by Reena's built-in receptionist. Reena identifies the caller ID as an existing patient and answers the phone with a personal greeting “Hello Mrs. Jones, how can we help today?” The patient wants to schedule an appointment. Reena recognizes it as an emergency and follows the practice's guidelines for emergency care as set forth in the configuration file or some other avenue.

Reena then looks for cross-sell opportunities—automatically accessing the caller's patient record simultaneously while conversing with the caller. For instance, the patient record indicates that it is time for a cleaning. It then suggests that the patient may want to schedule a cleaning while on the phone and take appropriate action as dictated by the patient and the office policies.

During the conversation, Reena asks if the patient's insurance information is correct. If it is, Reena checks the PMS to ensure that the practice takes the insurance (as described above), this action requires Reena to utilize a respective API to contact the insurance company to confirm that the patient is qualified and attempts to verify coverage. In its current state, dental insurance companies rarely allow API access to this kind of information. An individual must call the insurance company and speak to a customer service representative. There are often lengthy wait times, wasting large blocks of time for the dental office's staff. Reena dials the number and navigates the system to get to the appropriate source of information. If it is a human that answers, Reena can speak to the human on the other end of the phone in a human voice (using its IVR and AI technologies), verify the information and record it in the PMS. No staff time is required. Again, this action is handled simultaneously while conversing with the caller.

This use case can save thousands of hours over the course of a year and free up non-revenue producing staff. This allows the practice to streamline its headcount or redeploy the staff to more productive issues. In short, the present invention allows multitasking across multiple platforms and even with simultaneous actual live patient communications.

Efficiency is money and the present invention brings an efficiency improvement to respective healthcare industries at a historic, unexpected, and probably exponential level.

### Extension to Medical Healthcare

While optimized for dental workflows, the platform 10a may be extended to medical healthcare practices by substituting dental-specific vocabularies, entities, and workflow parameters with medical equivalents.

Accordingly, the invention provides a dental-first cloud-based conversational AI platform delivering technical improvements in administrative workflow automation, system interoperability, and patient care efficiency. The platform is extensible to broader medical healthcare applications, providing scalable, real-time, AI-driven solutions that satisfy USPTO PEG guidance for computer-implemented inventions.

It is understood that while certain forms of this invention have been illustrated and described, it is not limited thereto except insofar as such limitations are included in the following claims and allowable functional equivalents thereof.

1. A computer-implemented method for autonomously orchestrating healthcare workflow communications using artificial intelligence within a dental healthcare ecosystem, the method being executed by one or more processors in data communication with a network and comprising:
accessing, by the one or more processors, a conversational artificial intelligence agent comprising a natural language processing (NLP) subsystem and a speech processing subsystem;
receiving, via the speech processing subsystem, natural language input from a patient or practice user during a real-time interactive session;
processing the natural language input using the NLP subsystem to perform intent classification and entity extraction, thereby generating structured task data representing at least one requested healthcare-related action;
routing the structured task data to one or more external computer systems selected from the group consisting of a practice management system, an insurance verification interface, and a referral dental practice system;
autonomously executing, by the conversational artificial intelligence agent, one or more backend operations with the one or more external computer systems while maintaining the real-time interactive session with the patient or practice user; and
generating, based on results returned from the one or more external computer systems, a context-aware natural language response delivered to the patient or practice user;
wherein the method provides a technical improvement by reducing manual data entry, decreasing inter-system latency, and enabling concurrent execution of human-facing and machine-facing operations.

2. The method of claim 1, wherein the NLP subsystem comprises a transformer-based neural network architecture configured to perform joint intent classification and named-entity recognition using multi-head self-attention layers.

3. The method of claim 2, wherein the transformer-based neural network architecture is trained using a domain-adaptive training dataset comprising dental procedure terminology, scheduling workflows, insurance descriptors, and referral communications associated with dental practice operations.

4. The method of claim 1, wherein the conversational artificial intelligence agent is identified by a persistent agent identifier associated with a named virtual assistant persona.

5. The method of claim 1, wherein routing the structured task data to an insurance verification interface comprises autonomously navigating an interactive voice response (IVR) system using synthesized speech output and natural language understanding to obtain insurance eligibility or coverage information.

6. The method of claim 1, wherein routing the structured task data to a referral dental practice system comprises exchanging machine-readable semantic messages with a second conversational artificial intelligence agent associated with a referral dental practice.

7. The method of claim 6, further comprising coordinating an available appointment time by querying a scheduling module of the referral dental practice system and confirming the coordinated appointment time during the real-time interactive session.

8. The method of claim 1, wherein the conversational artificial intelligence agent concurrently maintains the real-time interactive session while executing the one or more backend operations using asynchronous task execution across distributed computing resources.

9. The method of claim 6, further comprising transmitting a HIPAA-compliant electronic patient record, including dental imaging or clinical notes, to the referral dental practice system upon confirmation of the referral appointment.

10. The method of claim 1, wherein the dental healthcare ecosystem is selectively extended to include at least one medical healthcare practice by applying a second domain-specific vocabulary, workflow model, and ontology associated with medical practice operations.

11. A cloud-based conversational artificial intelligence (AI) platform for a dental healthcare ecosystem, comprising:
one or more processors;
non-transitory memory in data communication with the one or more processors and configured for storing artificial intelligence program instructions;
at least one conversational AI agent executed by the one or more processors, the conversational AI agent comprising:
(i) a natural language processing (NLP) subsystem configured to transform unstructured dental-specific speech or text into structured task data; and
(ii) a speech processing subsystem configured to receive speech input and generate synthesized speech output; and
an orchestration engine executed by the one or more processors and operatively coupled to the at least one conversational AI agent,
wherein the orchestration engine is configured to autonomously execute dental administrative workflows across a plurality of cross-platform software systems based on the structured task data, thereby improving computer system efficiency and reducing manual administrative operations within the dental healthcare ecosystem.

12. The platform of claim 11, wherein the AI agent autonomously initiates inventory procurement for dental procedures.

13. The platform of claim 11, wherein each AI agent concurrently processes patient input and orchestrates backend tasks across multiple cross-platform systems without human intervention.

14. The platform of claim 11, wherein the orchestration engine manages error detection and recovery for API interactions with external dental systems.

15. The platform of claim 11, wherein each AI agent generates context-aware prompts to acquire missing information required to execute dental administrative tasks.

16. A cloud-based AI platform for a dental healthcare ecosystem, comprising:
a plurality of computing devices in data communication over a network;
a conversational AI agent deployed on each computing device, wherein each AI agent includes a natural language processing subsystem for interpreting and generating human language; and
an orchestration engine configured to coordinate real-time communications between AI agents and external systems, including practice management systems, insurance verification interfaces, and referral dental practice systems;
wherein the AI agents autonomously negotiate referral scheduling, exchange patient records, and verify dental procedure information while simultaneously maintaining conversational interactions with patients, providing a technical improvement over conventional manual coordination systems.

17. The platform of claim 16, wherein the orchestration engine is configured to decompose the structured task data into a plurality of workflow actions and to execute at least a subset of the workflow actions concurrently using asynchronous processing.

18. The platform of claim 16, wherein the orchestration engine maintains workflow state data identifying execution status, dependency conditions, and system responses associated with each workflow action.

19. The platform of claim 16, wherein the orchestration engine is further configured to invoke a plurality of application programming interfaces (APIs) associated with different healthcare software platforms and to route returned data to a conversational artificial intelligence agent for generation of a context-aware natural language response.
